(19) United States
(12) Patent Application Publication
(10) Pub. No.: US 2023/0018722 A1
(43) Pub. Date: Jan. 19, 2023

(54) Methods and System for Modulating Glycaemia

(71) Applicant: THE BIONICS INSTITUTE OF AUSTRALIA, East Melbourne (AU)

(72) Inventors: Sophie Morgan, East Melbourne (AU); Joel Villalobos, East Melbourne (AU); James Fallon, East Melbourne (AU)

(21) Appl. No.: 17/786,207

(22) PCT Filed: Dec. 16, 2020
(86) PCT No.: PCT/AU2020/051381

§ 371 Date: Jun. 16, 2022

(30) Foreign Application Priority Data
Dec. 17, 2019 (AU) 2019904772

(51) Int. Cl. A61N 1/36; A61N 1/372

(52) U.S. Cl. CPC A61N 1/36053; A61N 1/36171; A61N 1/36139; A61N 1/37247

(57) ABSTRACT

Methods and systems are disclosed for modulating glycaemia in a patient in which an activating stimulation signal is applied at an activating location at the vagus nerve, the activating stimulation signal configured to evoke a neural response in the vagus nerve; and a blocking stimulation signal is applied at a blocking location at the vagus nerve, the blocking stimulation signal configured to inhibit transmission of the evoked neural response along the vagus nerve past the blocking location; to produce unidirectional vagal nerve stimulation, the unidirectional vagal nerve stimulation being effective to modulate glycaemia in the patient.

### TECHNICAL FIELD

The present disclosure relates to application of electrical signals to the peripheral nervous system, and particularly to electrical stimulation of the vagus nerve for the purpose of modulating glycaemia.

### BACKGROUND

Diabetes mellitus (diabetes) is a metabolic disorder in which the body is unable to adequately regulate the level of glucose in the blood (glycaemia). If left untreated, diabetes can cause a range of serious long-term complications. Normally, glycaemia is regulated by the pancreas, which secretes hormones (insulin or glucagon) into the bloodstream to decrease or increase glucose concentration. Diabetes occurs when the pancreas does not produce enough insulin, or when the body is resistant to the insulin produced, resulting in abnormally high glucose levels in the blood. Conventional treatment of diabetes involves regulation of blood glucose levels by insulin injection and/or medication. Type I diabetic patients are generally insulin-dependent, whereas type II diabetics may require a range of medications. Such medications generally aim to lower blood glucose levels. However, many diabetic patients are refractory to existing medical therapies, or experience adverse side effects to the medications.

Neurostimulation is the purposeful modulation of activity in a subject's nervous system. Vagal nerve stimulation (VNS) has been found to have an effect on the release of insulin and glucagon and has been suggested as an alternative approach to the treatment of diabetes. However, to date, attempts to modulate glycaemia via VNS have resulted in an undesirable simultaneous increase of insulin and glucagon, resulting in an unwanted increase in blood sugar levels.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY OF THE DISCLOSURE

Aspects of the present disclosure provide a method for modulating glycaemia in a patient using vagal nerve stimulation (VNS). In general, the method comprises applying an activating stimulation signal configured to evoke a neural response in the vagus nerve and applying at least one blocking stimulation signal configured to inhibit transmission of the evoked neural response in one direction along the vagus nerve.

The activating stimulation signal may be applied at an activating location at the vagus nerve and the blocking stimulation signal may be applied at a blocking location at the vagus nerve. The blocking stimulation signal may be configured to inhibit transmission of the evoked neural response along the vagus nerve past the blocking location, to produce unidirectional stimulation of the vagus nerve.

Related to this, according to one aspect of the present disclosure, there is provided a method for modulating glycaemia in a patient, the method comprising:
applying an activating stimulation signal at an activating location at the vagus nerve, the activating stimulation signal configured to evoke a neural response in the vagus nerve; and
applying a blocking stimulation signal at a blocking location at the vagus nerve, the blocking stimulation signal configured to inhibit transmission of the evoked neural response along the vagus nerve past the blocking location,

to produce unidirectional vagal nerve stimulation, the unidirectional vagal nerve stimulation being effective to modulate glycaemia in the patient.

Throughout the specification and claims, the term glycaemia should be understood to mean the level of glucose in a patient's blood and may be considered interchangeable with the terms blood glucose concentration, blood glucose level or blood sugar level.

The evoked neural response may be understood to be an evoked compound action potential (ECAP) in the vagus nerve. Throughout the specification and claims, the term evoked compound action potential should be understood to refer to activation of a group of nerve fibers, but does not necessarily require activation of the whole nerve. While the evoked neural response may initially transmit along the vagus nerve in both the afferent and efferent directions, the blocking stimulation signal is configured to inhibit transmission in one of these directions, such that the evoked neural response transmits (or travels) along the nerve substantially in one direction only. As such, the unidirectional stimulation may be configured to be substantially afferent vagal nerve stimulation (aVNS) or substantially efferent vagal nerve stimulation (eVNS).

The unidirectional stimulation may be configured to be therapeutically effective to modulate endogenous hormone secretion of the patient, thereby to modulate glycaemia of the patient. For example, the unidirectional stimulation may be configured to be therapeutically effective to modulate insulin secretion, glucagon secretion and/or incretin hormone release in the patient, thereby to modulate glycaemia. The method may enable differential modulation of endogenous insulin secretion and endogenous glucagon secretion in the patient, thereby to modulate glycaemia. In some embodiments, the method may produce a simultaneous increase in insulin secretion and decrease in glucagon secretion, thereby to decrease glycaemia in the patient. Conversely, in some embodiments, the method may produce a simultaneous decrease in insulin secretion and increase in glucagon secretion, thereby to increase glycaemia in the patient.

The direction of the unidirectional stimulation may be selected based on the desired effect on the glycaemia of the patient. For example, aVNS may be selected to increase glycaemia, while eVNS may be selected to decrease glycaemia.

In some embodiments, the activating stimulation signal may have a frequency selected for activation of nerve fibres, including different types of nerve fibres. In some embodiments, the activating stimulation signal may have a frequency in the range of about 1 Hz to 50 Hz, or otherwise. For example, the frequency of the activating stimulation signal may be at least 1 Hz, at least 5 10 Hz, at least 20 Hz, at least 30 Hz, at least 40 Hz, or otherwise, and/or less than 50 Hz, less than 40 Hz, less than 30 Hz, less than 20 Hz, less than 10 Hz, or otherwise, or about 5 Hz, 10 Hz, 15 Hz, 20 Hz, 25 Hz, 30 Hz, 35 Hz, 40 Hz, 45 Hz, 50 Hz or more. The frequency of the activating stimulation signal may be configured to produce vagal nerve stimulation effective to modulate glycaemia in the patient.

The activating stimulation signal may have an amplitude configured to evoke a neural response in the vagus nerve. For example, the activating stimulation signal amplitude may be above a minimum threshold amplitude at which a compound action potential response is evoked in the vagus nerve (suprathreshold). Further, the activating stimulation signal amplitude may be below a maximum amplitude above which transmission of the evoked neural response is no longer effectively inhibited by the blocking stimulation signal, as discussed in further detail below. The amplitude of the activating stimulation signal may be configured to be within a therapeutic window defined between the minimum amplitude and maximum amplitude. In some embodiments, the activating stimulation signal may be configured to have an amplitude in the lower half of the therapeutic window.

In some embodiments, the amplitude of the activating stimulation signal may be configured to be within a predetermined range above, or at a predetermined level above, the minimum threshold amplitude. The configuring of amplitude of the activating stimulation signal to be within a predetermined range above, or at a predetermined level above, the minimum threshold amplitude, may be independent of any determination of the maximum threshold amplitude, for example. This may be advantageous if it is difficult to identify a maximum threshold amplitude (e.g. due to any noise effects associated with stimulation or otherwise). The amplitude of the activating stimulation signal may be configured to be within a predetermined range of 0 to 4 dB, 0 to 3 dB, 0.5 to 4 dB, 0.5 to 3.5 dB, 0.5 dB to 3 dB, 1 to 4 dB, or 1 to 3 dB, and/or at a predetermined level of about 1.5 dB, about 2 dB, or about 2.5 dB above the minimum threshold amplitude, for example.

In some embodiments, the method may comprise estimating an optimal dose of the activating stimulation signal. The optimal dose may include an optimal combination of amplitude and/or rate or duration of the activating stimulation signal. The estimated optimal dose may be confirmed, e.g. through receiving experimental data such as oral glucose tolerance test data carried out on the patient under no activating stimulation and then under activating stimulation at the estimated optimal dose. The activating stimulation signal with the estimated or confirmed optimal dose may be applied to the vagus nerve, and may have an amplitude within the therapeutic window, within the predetermined range, or at the predetermined level as discussed above.

The minimum and/or maximum threshold amplitudes may be determined based on an average of the respective threshold determined from a patient over an initial time period (e.g. 3 days). Alternatively, the minimum and/or maximum threshold amplitudes may be determined based on a moving average of the respective threshold as it changes over time (e.g. over months or years).

The blocking stimulation signal may be configured to inhibit transmission of the evoked neural response past the blocking location to produce a partial or full block of the vagus nerve at the blocking location.

The blocking stimulation may be configured to downregulate neural activity at the blocking location at the vagus nerve, for example, by increasing a threshold amplitude for generating a neural response in the vagus nerve at the blocking location. In such embodiments, the blocking stimulation signal may inhibit transmission of evoked neural responses past the blocking location when the evoked neural responses have an amplitude below the increased threshold amplitude. The degree of blocking may be defined by the increase in threshold amplitude. For example, the threshold amplitude may be increased by at least 0.5 dB, at least 1 dB, at least 2 dB, at least 3 dB or more. However, where the threshold amplitude is not measurably increased (that is, a 0 dB increase), blocking may still occur. The degree of blocking (or degree of inhibition) may be alternatively or additionally defined by the reduction of the amplitude of the evoked neural response transmitting past the blocking location. In some embodiments, the blocking stimulation signal may be configured to achieve a minimum degree of inhibition. For example, the blocking stimulation signal may be configured to achieve a reduction in evoked neural response amplitude of at least 50%, at least 60%, at least 70%, at least 75%, at least 80%, at least 85% at least 90%, at least 95% or more. In other embodiments, the blocking stimulation may inhibit transmission of all evoked neural responses past the blocking location, producing a full block.

In some embodiments, the blocking stimulation signal may inhibit the transmission of evoked neural responses and endogenous neural activity past the blocking location. In other embodiments, the blocking stimulation signal may inhibit the transmission of evoked neural responses while allowing the transmission of endogenous neural activity past the blocking location.

The applying of blocking stimulation may produce a nerve block which is reversible. For example, the inhibition of transmission of neural signals may persist only during application of the blocking stimulation signal (or, for a limited duration after cessation of the applying of the blocking stimulation signal). After cessation of the applying of the blocking stimulation signal, neural activity (whether evoked or endogenous) may transmit along the vagus nerve in substantially the same manner as before the blocking stimulation was applied. The applying of the reversible blocking stimulation signal in combination with the applying of the activating stimulation signal may advantageously allow for application of unidirectional stimulation of the vagus nerve without requiring transection of the nerve or inducing damage to the nerve.

In some embodiments, one or more parameters of the blocking stimulation signal may be selected to produce a localised (or focal) nerve block which inhibits transmission of the evoked neural response past the blocking location whilst allowing one or more adjacent regions of the nerve to conduct. In some embodiments, parameters of the blocking stimulation signal may be selected such that spread of the stimulation signal is limited to a small tissue volume (for example, by limiting spread of current due to a generated electric field). Additionally or alternatively, parameters of the blocking stimulation signal may be selected to produce a localised physiological effect in the vagus nerve rather than along the full length of the nerve. For example, the blocking stimulation signal may be configured to act on ion channels in proximity to the blocking location.

Providing a localised nerve block may eliminate (or at least minimise) the effect of the blocking stimulation signal at adjacent regions of the nerve. For example, the effect of the blocking stimulation may be negligible at the activating location where the activating stimulation signal is applied. This may advantageously allow the activating stimulation signal to be applied in close proximity to the blocking stimulation signal without inhibiting generation of the evoked neural response.

In some embodiments, the blocking stimulation signal may have a frequency in the range of about 1 kHz to about 150 kHz, about 10 kHz to about 100 kHz, about 10 kHz to about 50 kHz, about 10 kHz to about 40 kHz or otherwise. For example, the blocking stimulation signal may have a frequency of about 3 kHz, 4 kHz, 5 kHz, 10 kHz, 15 kHz, 20 kHz, 25 kHz, 30 kHz, 35 kHz 40 kHz, 45 kHz, 50 kHz, 60 kHz, 70 kHz, 80 kHz, 90 kHz, 100 kHz, 110 kHz, 120 kHz, 130 kHz, 140 kHz, 150 kHz or otherwise. The frequency of the blocking stimulation signal may be selected to be high enough to produce an effective local block and below an upper limit which would ablate or otherwise damage the nerve.

In some embodiments, the blocking stimulation signal may comprise a pulsed signal. For example, in some embodiments, the blocking stimulation signal may comprise a square pulse waveform. In other embodiments, the blocking stimulation signal may comprise a sinusoidal waveform. In still other embodiments, the blocking stimulation may comprise a ramp, triangle or sawtooth waveform, an exponential waveform or other waveform shapes.

In some embodiments, a plurality of blocking stimulation signals may be applied at a corresponding plurality of blocking locations at the vagus nerve. In some embodiments, the blocking stimulation signals may be applied at two, three, four or more blocking locations on the vagus nerve. For example, the activating stimulation signal may be applied at an activating location and blocking stimulation signals applied at first and second blocking locations at the vagus nerve. The blocking stimulation signals may be substantially identical, or they may be different, to each other. For example, a blocking stimulation signal comprising a sinusoidal waveform may be applied at a first blocking location while a blocking stimulation signal comprising a square pulsed waveform may be applied at a second blocking location. Alternatively, a blocking stimulation signal comprising a square pulsed waveform or sinusoidal waveform may be applied at each blocking location.

In some embodiments, the plurality of blocking stimulation signals may be applied at the plurality of blocking locations simultaneously. Applying the blocking stimulation signals simultaneously may increase the effectiveness of the nerve block (for example, by increasing the range of the therapeutic window). Alternatively, the plurality of blocking stimulation signals may be applied at the plurality of blocking locations at different times.

Applying blocking stimulation signals at a plurality of discrete blocking locations may advantageously improve the safety of the blocking stimulation e.g. by reducing the amplitude of the blocking stimulation required at each location (and therefore decreasing the likelihood of damage to the nerve due to applying the blocking stimulation signal at one blocking location only).

In some embodiments, the blocking stimulation signal has an amplitude (as measured peak-to-peak) of between about 1 mA to about 50 mA or otherwise. For example, the blocking stimulation signal may have an amplitude of at least 5 10 mA, at least 20 mA, at least 30 mA, at least 40 mA, or otherwise, and/or less than 50 mA, less than 40 mA, less than 30 mA, less than 20 mA, less than 10 mA, or otherwise, or about 1 mA, 5 mA, 10 mA, 20 mA, 30 mA, 40 mA, 50 mA or otherwise.

In some embodiments, the method may further comprise detecting the evoked neural response in the vagus nerve. In some embodiments, one or more parameters of at least one of the activation and blocking signals may be at least partially based on properties of the detected neural response. For example, one or more of a stimulus frequency, stimulus amplitude and stimulus duration for the activating and/or blocking stimulation signal may be configured based on the detected neural response.

The detecting may confirm the effectiveness (or otherwise) of the activating stimulation signal in evoking a neural response in the vagus nerve. For example, the detecting may be performed at the same time as applying the activating stimulation signal, but in the absence of a blocking stimulation signal, to determine a minimum amplitude of the activating stimulation signal at which a neural response is evoked in the vagus nerve.

The detecting may further confirm the effectiveness of the blocking stimulation signal. That is, the detecting may be performed at the same time as applying the blocking and activating stimulation signals to determine the degree to which transmission of the evoked neural response past the blocking location is inhibited. In some embodiments, the detecting may be used to determine a maximum amplitude of the activating stimulation signal at which the evoked neural response is effectively blocked by the blocking stimulation signal. For example, the detecting may be performed at a detecting location on the vagus nerve, where the blocking location is positioned between the activating and detecting locations. Detection of an evoked neural response at the detecting location may indicate that transmission of the evoked neural response past the blocking location was not completely blocked by the blocking stimulation signal. In such instances, detection of the evoked neural response may indicate, or be used to determine, a degree of inhibition of the evoked neural response. Alternatively, lack of detection of an evoked neural response at the detecting location may indicate that transmission of the evoked neural response past the blocking location was completely blocked by the blocking stimulation signal. Detection of the evoked neural response may be used to determine or confirm a minimum threshold amplitude, a maximum threshold amplitude, and/or a therapeutic window (defined between the minimum threshold amplitude and maximum threshold amplitude) where unidirectional VNS may be achieved.

In some embodiments, the method may further comprise detecting glycaemia of the patient. Glycaemia may be detected by a glucose sensor, such as a continuous glucose monitoring system or a blood glucose meter, for example. In some embodiments, one or more parameters of at least one of the activation and blocking signals may be at least partially based on properties of the detected glycaemia of the patient.

The method may further comprise selectively triggering the applying of the activating stimulation signal and the blocking stimulation signal in response to the detected glycaemia. Moreover, the locations at which the activating stimulation signal and the blocking stimulation signal are applied may be selected to achieve unidirectional vagal nerve stimulation that is afferent vagal nerve stimulation when the detected glycaemia is lower than a predetermined lower limit. Alternatively or additionally, the locations at which the activating stimulation signal and the blocking stimulation signal are applied may be selected to achieve unidirectional vagal nerve stimulation that is efferent vagal nerve stimulation when the detected glycaemia is higher than a predetermined upper limit. The lower and upper limits may be predetermined, for example, by a healthcare representative of the patient.

By selectively applying stimulation in response to glycaemia of the patient, the stimulation can be applied only when needed. This may reduce the likelihood of damage to the nerve. In some embodiments, the method may comprise closed-loop glycaemic control to ameliorate glycaemic variability in the patient.

Alternatively or additionally, the method may comprise monitoring food intake of the patient and selectively triggering the applying of the activating stimulation signal and the blocking stimulation signal in response to food intake of the patient. For example, the patient (or the patient's healthcare representative or carer) may trigger application of the activating and blocking stimuli configured to produce eVNS either shortly before, during or after ingesting a meal. The eVNS may be configured to persist for a predetermined period of time to modulate (by lowering glycaemia) the peak in glycaemia due to the meal. In some embodiments, one or more parameters of the activating and/or blocking stimulation may be configured based on the content of the meal.

The activating and blocking locations may be at a sub-diaphragmatic portion of the vagal nerve. In some embodiments, the activating and blocking locations may be at the abdominal vagus nerve.

In some embodiments, the patient may have a condition or disease associated with impaired glucose regulation. The method may be used for treating or preventing a condition or disease associated with impaired glucose regulation in the patient. For example, the method may be used for treating or preventing type 2 diabetes in the patient.

According to another aspect of the disclosure, there is provided a system configured to perform the above described method, the system comprising:

a first pair of electrodes selectively operable for applying the activating stimulation signal; and

a second pair of electrodes selectively operable for applying the blocking stimulation signal.

According to another aspect of the disclosure, there is provided a system configured to modulate glycaemia in a patient, the system comprising:

a first pair of electrodes selectively operable for applying an activating stimulation signal to the vagus nerve, the activating stimulation signal configured to produce an evoked neural response in the vagus nerve; and

a second pair of electrodes selectively operable for applying a blocking stimulation signal to the vagus nerve, the blocking stimulation signal configured to inhibit transmission of the evoked neural response along the vagus nerve past the second pair of electrodes.

In some embodiments, the system may comprise a third pair of electrodes. The third pair of electrodes may be selectively operable as a pair of detecting electrodes for detecting the evoked neural response.

In some embodiments, each pair of electrodes is comprised in an electrode array. The electrode array may be adapted for placement on the vagus nerve such that the electrodes of at least one of the electrode pairs are positioned on opposite sides of the nerve. Electrode placement on opposite sides of the nerve may advantageously improve the efficacy of the applied stimulation signals. For example, electrode placement on opposite sides of the nerve may increase the effectiveness of the blocking stimulation signal in inhibiting transmission of the evoked neural response. Further, electrode positioning on opposite sides of the nerve may confine the spread of the blocking stimulation signal to a smaller tissue volume than if the electrodes of the electrode pair were placed side by side on the nerve. This may advantageously contribute to the creation of a focal nerve block.

The first and second pairs of electrodes may be spaced from each other by a distance A. The second pair of electrodes may be located intermediate the first and third pairs of electrodes. The third pair of electrodes may be spaced from the second pair of electrodes by a distance B. The distances A and B may be substantially equal, i.e., it may be that A=B, or they may be different. The distance A (and/or B) may be configured to be large enough such that application of the blocking stimulation signal at one pair of electrodes does not inhibit generation of an evoked neural response at another pair of electrodes.

The first, second and optionally third pairs of electrodes may be in a substantially fixed relationship. The relative orientation and/or location of the pairs of electrodes may therefore be substantially pre-defined, rather than being selected by a surgeon.

In some embodiments, each of the first, second and optionally third pairs of electrodes (also referenced herein as the first, second and third electrode pairs) may be selectively operable for applying the activating stimulation signal, for applying the blocking stimulation signal or for detecting the evoked neural response.

In accordance with the above, in one embodiment, the first pair of electrodes is configured for applying the activating stimulation signal at an activating location at the vagus nerve, the second pair of electrodes is configured for applying the blocking stimulation signal at a blocking location at the vagus nerve and the third pair of electrodes is configured for detecting the evoked neural response at a detecting location at the vagus nerve.

Nevertheless, the different pairs of electrodes may be selectively operable in different configurations. This may advantageously enable selection of the direction of the unidirectional stimulation. For example, the direction of the unidirectional stimulation may be selected by appropriately configuring the relative positions of the electrodes for applying the activating stimulation signal and the blocking stimulation signal.

The provision of first, second and third electrode pairs does not preclude the provision of fourth, fifth or yet further electrode pairs, whether for applying activating or blocking stimulation signals and/or detecting evoked neural responses. As one example, four electrode pairs may be provided. The second pair of electrodes may be a selectively operable for applying an activating stimulation signal to the vagus nerve, the third pair of electrodes may be selectively operable for applying a blocking stimulation signal to the vagus nerve and the remaining two (e.g. first and fourth) electrode pairs may be selectively operable for detecting an evoked neural response at the vagus nerve in different directions (i.e. at respective first and second detecting locations). The first pair of electrodes may monitor the evoked neural response in a first direction (e.g. in an efferent direction) and the fourth pair of electrodes may monitor the electrical response in a second direction (e.g. in an afferent direction).

Alternatively or additionally, a plurality of electrode pairs may be configured to apply the blocking stimulation signal (or a plurality of blocking stimulation signals) at a corresponding plurality of blocking locations on the vagus nerve.

In some embodiments, at least one of the electrode pairs is comprised in an electrode mounting device. The electrode mounting device may be adapted to mount to the vagus nerve to electrically interface the at least one electrode pair with the vagus nerve. In some embodiments, the electrode mounting device is adapted to clamp to the vagus nerve. In some embodiments, the mounting device comprises at least one cuff, the electrodes of each of the electrode pairs being positioned on opposite sides of the cuff. In some embodiments, the mounting device comprises a single elongate cuff, each electrode pair of the electrode array being mounted in the cuff. In some embodiments, the cuff comprises a pair of cuff portions adapted to mount to the vagus nerve. The cuff portions, when in a closed configuration, may together define an inner surface with a semi-elliptical, semi-oblong or semi-rectangular profile to contact an outer surface of the vagus nerve. In some embodiments, at least one of the cuff portions comprises a recess or channel extending along the cuff and adapted to receive a portion of the vagus nerve therein.

In some embodiments, the electrode array may comprise an anchor adapted to provide mechanical stability to the array. The anchor may comprise a projecting member, such as a tab. In one embodiment, the anchor comprises a tab primarily formed from silicone. The anchor may comprise additional materials for improved strength or structural integrity. In one embodiment, the anchor comprises a silicone tab embedded with Dacron (polyethylene terephthalate) fibres. The anchor may be adapted to be connected to a surrounding physiological structure. For example, in some embodiments, the anchor is adapted to be sutured to the oesophagus.

The electrode array may comprise or may be connected to one or more electrical signal generators that generate the electrical stimulation signals to apply, via the respective pair(s) of electrodes, to the vagus nerve. The electrode array may comprise or may be connected to one or more monitoring and/or recording devices that receive, from the respective pair(s) of electrodes, detected neural response signal(s) and process the detected neural response signal(s).

The system may further comprise a glucose sensor adapted to detect glycaemia of the patient. For example, the glucose sensor may be a continuous glucose monitoring system or a blood glucose meter.

The system may further comprise a controller for adjusting one or more parameters of the activating and blocking stimulation signals and/or selectively triggering the applying of the activating and blocking stimulation signals. The system controller may be configured to trigger the applying of the activating and blocking stimulation signals in response to one or more of: an input from the patient, a caretaker or a clinician; and detected glycaemia of the patient. The system controller may be configured to adjust one or more parameters of the activating and blocking stimulation signals in response to one or more parameters of the evoked neural response and/or detected glycaemia of the patient.

In some embodiments, selectively triggering the applying of the activating and blocking stimulation signals may comprise selectively triggering the applying of afferent vagal nerve stimulation (aVNS) or efferent vagal nerve stimulation (eVNS).

The system may comprise a patient interface that is configured to receive the input from the patient, caretaker or clinician to selectively trigger the aVNS stimulation or the eVNS stimulation. In some embodiments, the patient interface may also be configured to receive an input from the patient, caretaker or clinician to cease applying of the activating and/or blocking stimulation signals.

The patient interface may comprise one or more buttons to receive the input(s) from the patient, caretaker or clinician. The patient interface may be a hand-held battery-powered unit. The patient interface may be configured to be held on a person's body. For example, the patient interface may include a clip for clipping to clothing. In some embodiments, the patient interface may be configured to mount on a key ring, and may therefore have a key ring loop. The patient interface may communicate via wires or wirelessly with the system controller, or may be integrated into the system controller.

As indicated, in addition or as an alternative to patient input, selectively triggering the applying of the activating and blocking stimulation signals may be in response to glycaemia of the patient detected by a glucose sensor. Moreover, adjusting one or more parameters of the activating and blocking stimulation signals may be in response to one or more parameters of the evoked neural response and/or detected glycaemia of the patient. In this regard, the glucose sensor may detect hypoglycaemia and the system controller may selectively trigger aVNS stimulation, or reduce or stop eVNS stimulation, to increase blood glucose levels in the patient and/or the glucose sensor may detect hyperglycaemia and the system controller may selectively trigger eVNS stimulation, or reduce or stop aVNS stimulation, to decrease blood glucose levels in the patient. Alternatively, the glucose sensor may detect hypoglycaemia and the system controller may cause issuance of an alert to a patient, caretaker or clinician to selectively trigger aVNS stimulation to increase blood glucose levels in the patient using the patient interface, and/or the glucose sensor may detect hyperglycaemia and the system controller may cause issuance of an alert to a patient, caretaker or clinician to selectively trigger eVNS stimulation to decrease blood glucose levels in the patient. The alert may be an alert signal (e.g. audible, tactile or visible indicator) provided at the patient interface.

The system controller may be configured to identify a minimum threshold amplitude at which a compound action potential response is evoked in the vagus nerve and/or a maximum threshold amplitude above which transmission of the evoked neural response is not effectively inhibited by the blocking stimulation signal.

The system controller may be configured to apply the activating stimulation signal having an amplitude that is within a therapeutic window defined between the minimum threshold amplitude and the maximum threshold amplitude (e.g. within the lower half of the therapeutic window), and/or within a predetermined range above, or at a predetermined level above, the minimum threshold amplitude, e.g. in accordance with amplitude levels and ranges discussed above.

In some embodiments, the system controller may estimate an optimal dose of the activating stimulation signal. The optimal dose may include an optimal combination of amplitude and/or rate or duration of the activating stimulation signal. The estimated optimal dose may be confirmed, e.g. through receiving experimental data such as oral glucose tolerance test data carried out on the patient under no activating stimulation and then under activating stimulation having the estimated optimal dose. The activating stimulation signal with the estimated or confirmed optimal dose may be applied to the vagus nerve, and may have an amplitude within the therapeutic window, within the predetermined range, or at the predetermined level as discussed above.

The system controller may be at least partially implantable (e.g. subcutaneously) in the patient. The patient interface, if provided, may be external to the patient. The system controller may be connected to the electrode array via a lead. The lead may be disconnectable from the system controller e.g., to assist with storage of system components or the implantation process.

The system may further comprise a diagnostics unit, which may be provided as a software application on a desktop computer, laptop, tablet, smartphone or otherwise. The diagnostics unit may be configured to receive, store and display data from the system controller relating to the applied stimulation parameters, glycaemia of the patient over time, therapeutic outcomes or otherwise.

Generally, it will be recognised that the system controller can comprise a number of control or processing modules for controlling one or more components or functions of the system and may also include one or more storage devices, for storing data, such as data relating to the parameters of activating and/or blocking stimulation signals, including any one or more of the minimum threshold amplitude, the maximum threshold amplitude, or the predetermined therapeutic window, for example. The modules and storage devices can be implemented using one or more processing devices and one or more data storage units, which modules and/or storage devices may be at one location or distributed across multiple locations and interconnected by one or more communication links.

Further, the modules can be implemented by a computer program or program code comprising program instructions. The computer program instructions can include source code, object code, machine code or any other stored data that is operable to cause the processor to perform the steps described. The computer program can be written in any form of programming language, including compiled or interpreted languages and can be deployed in any form, including as a stand-alone program or as a module, component, subroutine or other unit suitable for use in a computing environment. The data storage device(s) may include a non-transitory computer-readable memory medium comprising instructions that cause the processor to perform steps as described herein. The data storage device(s) may include suitable computer readable media such as volatile (e.g., RAM) and/or non-volatile (e.g., ROM, disk) memory or otherwise.

According to another aspect of the disclosure, there is provided use of the above described system to produce substantially unidirectional vagal nerve stimulation, the unidirectional vagal nerve stimulation being effective to modulate glycaemia in the patient.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments of the present disclosure are now described with reference to the accompanying drawings in which:

FIG. 1 shows a flowchart of steps carried out in a method of stimulating the vagus nerve to modulate glycaemia in a patient, according to an embodiment of the present disclosure;

FIG. 2 shows an electrode array according to an embodiment of the present disclosure configured to perform the method of FIG. 1, along with associated activating and blocking stimulation signal traces and detected neural response trace;

FIG. 3 shows another embodiment of an electrode array according to the present disclosure configured to perform the method of FIG. 1;

FIG. 4 shows traces of neural responses evoked during application of an activating stimulation signal alone (left) and with application of a blocking stimulation signal (right) and a therapeutic window for directional stimulation (indicated by the dashed line);

FIG. 5A shows a system diagram of apparatus according to an embodiment of the present disclosure configured to perform the method of FIG. 1;

FIG. 5B also shows a system diagram of apparatus according to an embodiment of the present disclosure configured to perform the method of FIG. 1;

FIG. 5C also shows a system diagram of apparatus according to an embodiment of the present disclosure configured to perform the method of FIG. 1;

FIG. 6 shows an example of the effect of eVNS according to the method of FIG. 1 on glucose and pancreatic hormone (glucagon and insulin) secretions in normal rats;

FIG. 7 shows an example of the effect of eVNS according to the method of FIG. 1 on incretin hormone secretions (GLP-1) in normal rats;

FIG. 8 shows an example of the effect of eVNS according to the method of FIG. 1 on blood glucose levels in chemically-induced diabetic rats;

FIG. 9 shows an example of the effect of eVNS according to the method of FIG. 1 on blood glucose levels in diet-induced diabetic rats;

FIG. 10 shows an example of the effect of a sinusoidal wave blocking stimulation signal on the threshold for evoking a neural response in the vagus nerve of a rat;

FIG. 11 shows an example of the effect of a square pulse blocking stimulation signal on the threshold for evoking a neural response in the vagus nerve of a rat; and

FIG. 12 shows a flowchart of steps carried out to identify an optimal amplitude of the activating stimulation signal for applying to a vague nerve, according to an embodiment of the present disclosure.

### DETAILED DESCRIPTION

Methods for modulating glycaemia of a patient using vagal nerve stimulation (VNS) according to embodiments of the present disclosure are now described.

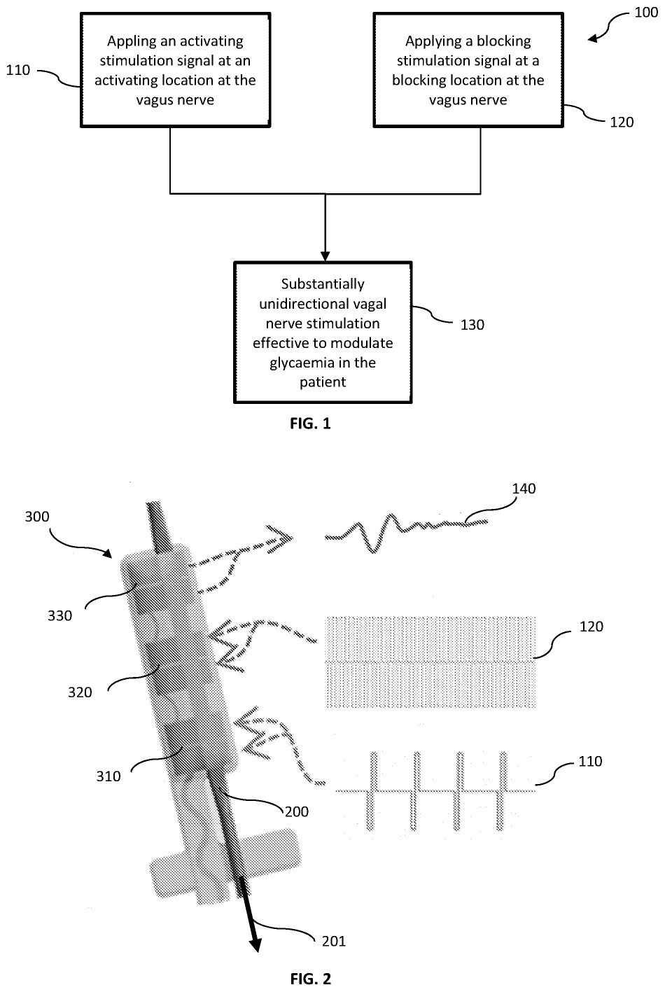

Referring to flowchart 100 of FIG. 1, a method for modulating glycaemia in a patient according to an embodiment of the present disclosure is illustrated. The method comprises applying an activating stimulation signal 110 at an activating location at the vagus nerve and applying a blocking stimulation signal 120 at a blocking location at the vagus nerve. The activating stimulation signal 110 is configured to evoke a neural response in the vagus nerve, and the blocking stimulation signal 120 is configured to inhibit transmission of the evoked neural response along the vagus nerve past the blocking location, to produce unidirectional vagal nerve stimulation 130. The unidirectional vagal nerve stimulation 130 is effective to modulate glycaemia in the patient.

FIG. 2 shows example signal traces for the activating stimulation signal 110 and the blocking stimulation signal 120, respectively. As can be seen, in this embodiment, the blocking stimulation signal 120 generally has a higher frequency than activating stimulation signal 110. For example, in one embodiment, the activating stimulation signal 110 has a frequency of about 15 Hz, while the blocking stimulation signal has a frequency of about 4 kHz or about 40 kHz.

In some embodiments, the method may further comprise detecting the evoked neural response 140. FIG. 2 shows an example detected neural response 140 (or evoked compound action potential, ECAP).

As shown in the example of FIG. 2, the activating and blocking stimulation signals 110 and 120 may be applied to the vagus nerve 200 by respective first and second pairs of electrodes 310 and 320 comprised in an electrode array 300. The first and second electrode pairs 310, 320 interface with the vagus nerve 200 at respective first and second locations. The electrode array optionally comprises a third pair of electrodes 330 which interfaces the vagus nerve 200 at a third location. The electrode array is not limited to three pairs of electrodes, and may further include fourth, fifth, sixth or more pairs of electrodes interfacing the vagus nerve at respective fourth, fifth, sixth or more locations.

The blocking stimulation signal 120 is configured to inhibit transmission of a neural response evoked by the activating stimulation 110 past the blocking location. As such, in the example of FIG. 2, when the blocking stimulation signal and activating stimulation signal are simultaneously applied, the evoked neural response will transmit in substantially one direction only along the nerve (in this case, down the nerve 200 in the direction of the arrow 201).

Each of the first and second and third electrode pairs 310, 320, 330 may be selectively operable for applying the activating stimulation signal 110, for applying the blocking stimulation signal 120 or for detecting the evoked neural response 140. As such, the relative locations at which the activating and blocking stimulation signals are applied to the vagus nerve may be selected depending on the desired direction of stimulation along the vagus nerve 200. For example, if the second (that is, the middle) electrode pair 320 is selected for applying the blocking stimulation, the direction of the vagal nerve stimulation (VNS) may be determined by whether the proximal or distal pair or electrodes is selected for applying the activating stimulation. For example, (depending on the orientation in which the electrode array 300 is mounted to the nerve 200) the activating stimulation signal 110 may be applied at the first electrode pair 310 to achieve eVNS, or at the third electrode pair 320 to achieve aVNS.

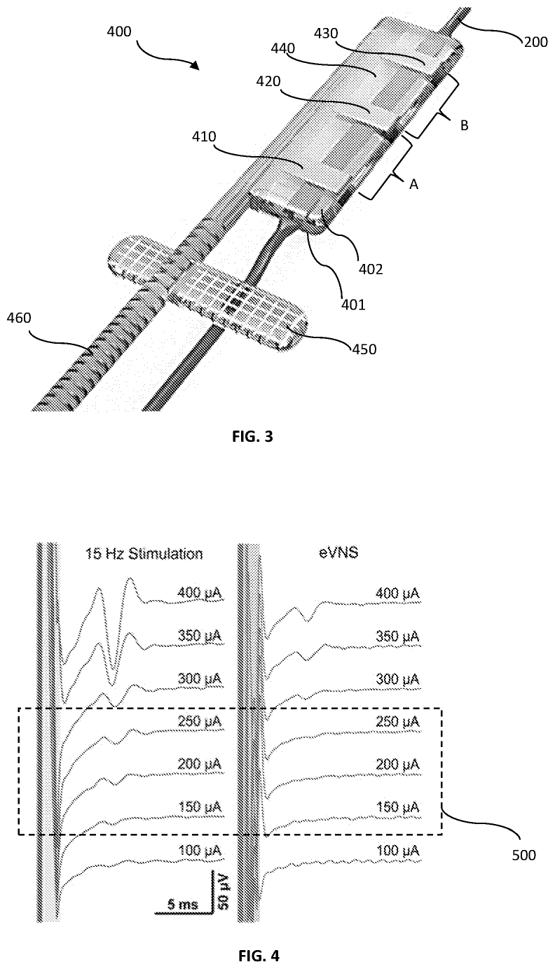

FIG. 3 shows an electrode array 400 according to another embodiment of the present disclosure, suitable for use in the above described method. The array comprises first, second and third electrode pairs 410, 420, 430, the second electrode pair 420 being disposed intermediate the first and third electrode pairs 410, 430. Each of the electrode pairs is comprised in an electrode mounting device in the form of a cuff 440. The cuff 440 is adapted to mount to the vagus nerve 200 to electrically interface the electrode pairs 410, 420, 430 with the vagus nerve 200. The cuff 440 comprises a pair of cuff portions 401, 402 adapted to be brought together to clamp to opposite sides of the nerve 200. The cuff portions 401, 402, when in a closed configuration, may together define an inner surface with a semi-elliptical, semi-oblong or semi-rectangular profile to contact an outer surface of the vagus nerve. In some embodiments, at least one of the cuff portions 401, 402 comprises a recess or channel extending along the cuff and adapted to receive a portion of the vagus nerve therein.

In some embodiments, the cuff portions 401, 402 may be sutured or otherwise fixed together after clamping to limit movement of the array 300. One electrode of each of the electrode pairs 410, 420, 430 is disposed in each cuff portion 401, 402. The cuff 440 is adapted for placement on the vagus nerve 200 such that, when the cuff 440 is mounted to the nerve 200, the electrodes of each of the electrode pairs 410, 420, 430 are positioned on opposite sides of the nerve 200.

As shown in FIG. 3, the second pair of electrodes 420 is situated intermediate the first and third pairs of electrodes 410, 430. The first and second pairs of electrodes 410, 420 are separated from each other by a distance A, while the second and third pairs of electrodes are separated from each other by a distance B. In the illustrated embodiment, the distances A and B are substantially equal. In other embodiments, the distances A and B may be different to each other. The distances A and/or B may be configured such that the electrode pair selected for applying the activating stimulation signal 110 is sufficiently spaced from the electrode pair selected for applying the blocking stimulation 120, such that generation of evoked neural responses at the activating location is not inhibited by the blocking stimulation applied at the blocking location.

It will be appreciated that the method of the invention may be applied using alternative electrode arrays, different to those shown in FIGS. 2 and 3. For example, one or more of the electrode pairs may be comprised in separate cuffs, separate mounting devices or otherwise. This may allow the spacing between the electrode pairs to be adjusted, for example.

Again, each of the first, second and third electrodes 410, 420 and 430 are selectively operable for applying the activating stimulation signal 110, for applying the blocking stimulation signal 120 or for detecting the evoked neural response 140.

In some embodiments, a plurality of blocking stimulation signals 120 may be applied at a corresponding plurality of blocking locations via more than one pair of electrodes. Applying blocking stimulation signals at more than one location may advantageously increase the effectiveness of the blocking. For example, in the embodiment of FIG. 3, the activating stimulation signal may be applied at the first pair of electrodes 410 while blocking stimulation signals are applied at both the second and third pairs of electrodes 420 and 430. The distance B between the electrodes may be configured to ensure that the block produced at each of the second and third pairs of electrodes 420 and 430 are independent. It will be appreciated that, in this embodiment, all three pairs of electrodes 410, 420 and 430 are utilised for applying either the activating or blocking stimulation signals 110, 120. As such, there is no electrode pair available for detecting the evoked neural response 140. In other embodiments, the electrode array 400 may comprise fourth, fifth, sixth or more pairs of electrodes, potentially allowing for applying of activating and/or blocking stimulation signals 110, 120 at multiple activating or blocking locations and/or detecting evoked neural responses 140 at multiple detecting locations.

In the embodiment of FIG. 3, the electrode array comprises an anchor in the form of a projecting tab 450, adapted to provide mechanical stability to the array by connecting to a surrounding physiological structure. The tab 450 may be formed substantially of silicone and may comprise additional reinforcing material. The electrodes are each electrically connected to a cable 460.

The frequency of the blocking stimulation signal 120 may be selected to downregulate neural activity at the blocking location at the vagus nerve 200, for example, by increasing a threshold amplitude for generating a neural response in the vagus nerve 200 at the blocking location. In such embodiments, the blocking stimulation signal may inhibit transmission of evoked neural responses past the blocking location when the evoked neural responses have an amplitude below the increased threshold amplitude.

FIG. 4 illustrates example detected evoked neural responses, confirming the effectiveness of the activating stimulation and blocking stimulation. The threshold at which ECAPs were generated using a 15 Hz activating stimulation signal can be seen in the left-hand pane at 150 μA. As shown in the right-hand pane, when the same activating stimulation signal was applied in combination with a 40 kHz, 8 mA blocking stimulation signal, the threshold is increased to 300 μA. The blocking stimulation signal can therefore be seen to downregulate the activity of the vagus nerve to at least inhibit transmission of the evoked neural response. Depending on the amplitude of the evoked neural response, the blocking stimulation may inhibit transmission of the response to different degrees. The level of inhibition may be defined by a percentage reduction in the amplitude of the evoked neural response.

In some embodiments, the blocking stimulation signal may comprise a square pulsed waveform. In other embodiments, the blocking stimulation signal may comprise a sinusoidal waveform.

Methods according to the present disclosure may not require complete blocking of the evoked response. Nevertheless, an effective level of blocking may be defined by a minimum percentage reduction in response amplitude. As shown in FIG. 4 a therapeutic window 500 for the amplitude of the activating stimulation signal may be defined between a minimum (original threshold) and maximum (increased threshold) amplitude which is able to generate ECAPs while also being effectively blocked. In practice, the activating stimulation signal may be configured to be within the lower half of the defined therapeutic window. This may increase the likelihood that transmission of the evoked neural response is completely blocked by the blocking stimulation signal.

In some embodiments, rather than identifying and locating the amplitude of the activating stimulation signal within a therapeutic window partially defined by a maximum amplitude, the amplitude of the activating stimulation signal may be configured to be within a predetermined range above, or at a predetermined level above, the minimum threshold amplitude. For example, as may be identified in Examples below, it may be optimal to set the amplitude of the activating stimulation signal to be within a predetermined range above the minimum threshold amplitude of 0 to 4 dB or at a predetermined level above the minimum threshold amplitude of about 2 dB, although other ranges and levels may be used, e.g. as discussed elsewhere in the present description.

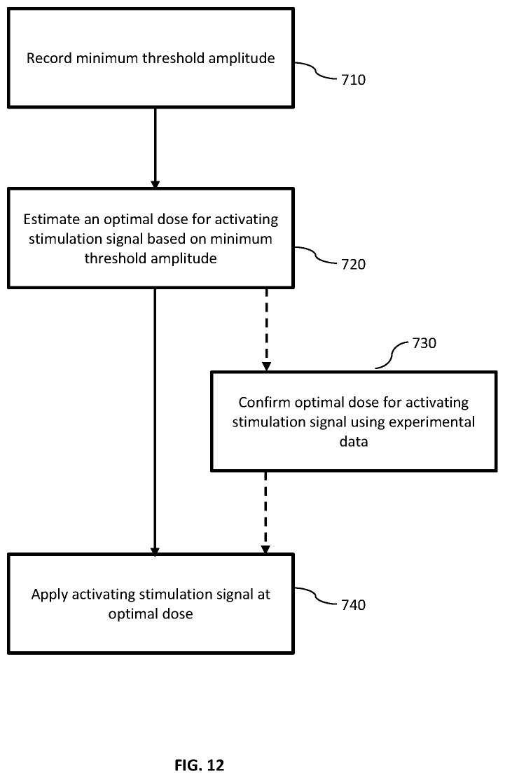

FIG. 12 shows a flowchart of steps carried out, e.g. at least partially by a system controller, to identify a suitable amplitude of the activating stimulation signal for applying to a vague nerve. At 710, a minimum threshold amplitude of the activating stimulation signal to evoke a compound action potential response in the vagus nerve is recorded. At 720, an optimal dose of the activating stimulation signal based on the minimum threshold amplitude (e.g. within 0 to 4 dB or about 2 dB above the minimum threshold), and with a suitable rate/duration, is estimated. At 730, the estimated optimal dose is optionally confirmed, e.g., through receiving experimental data such as oral glucose tolerance test data carried out on the patient under no activating stimulation and then under activating stimulation at the estimated optimal dose. At 740, the controller is configured to apply the activating stimulation signal with the estimated or confirmed optimal dose to the vagus nerve.

The minimum threshold amplitude may be determined at 710 based on an average of the threshold over an initial time period (e.g. 3 days). Alternatively, the minimum threshold amplitude may be determined based on a moving average of the threshold as it changes over time (e.g. over months or years).

The blocking effect of the blocking stimulation signal is configured to be localised (or focal) to the blocking location on the vagus nerve at which the blocking stimulation signal is applied (that is, local to the electrode pair used for applying the blocking stimulation signal). The blocking stimulation signal parameters may therefore be configured such that the current spread is limited to a relatively small volume of tissue and such that only ion channels in close proximity to the blocking electrodes are affected. As such, the blocking stimulation signal does not inhibit generation of ECAPs at adjacent locations on the nerve.

In embodiments of the present disclosure, detection of evoked neural responses may be used for an initial setup, calibration or tuning of the activating stimulation signal and blocking stimulation signal parameters. For example, the activating stimulation signal may be initially applied in the absence of a blocking stimulation signal and any resultant neural response detected. One or more parameters of the activating stimulation signal may be adjusted based on the detected neural response. For example, if no neural response is detected, parameters of the activating stimulation signal may be adjusted (for example, the amplitude of the activating stimulation signal may be increased) to achieve reliable generation of an evoked neural response. The activating stimulation signal may be subsequently applied in combination with the blocking stimulation signal and any resultant neural response detected.

In embodiments where the detecting is performed at a detecting location on the vagus nerve, where the blocking location is positioned between the activating and detecting locations (for example, as shown in FIG. 2), the detecting may confirm the effectiveness of the blocking stimulation signal. For example, detection of an evoked neural response at the detecting location may indicate that transmission of the signal past the blocking location was not completely blocked by the blocking stimulation signal. In such instances, the degree of blocking may be quantified (for example, by a percentage reduction in amplitude of the evoked response). Alternatively, the absence of a detected neural response at the detecting location may indicate that transmission of the evoked neural response past the blocking location was completely blocked by the blocking stimulation signal.

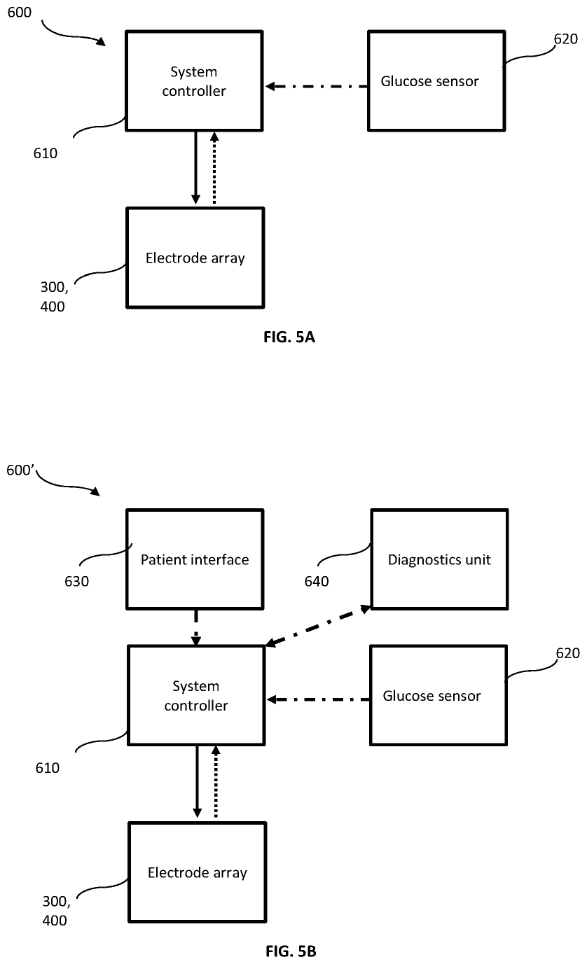

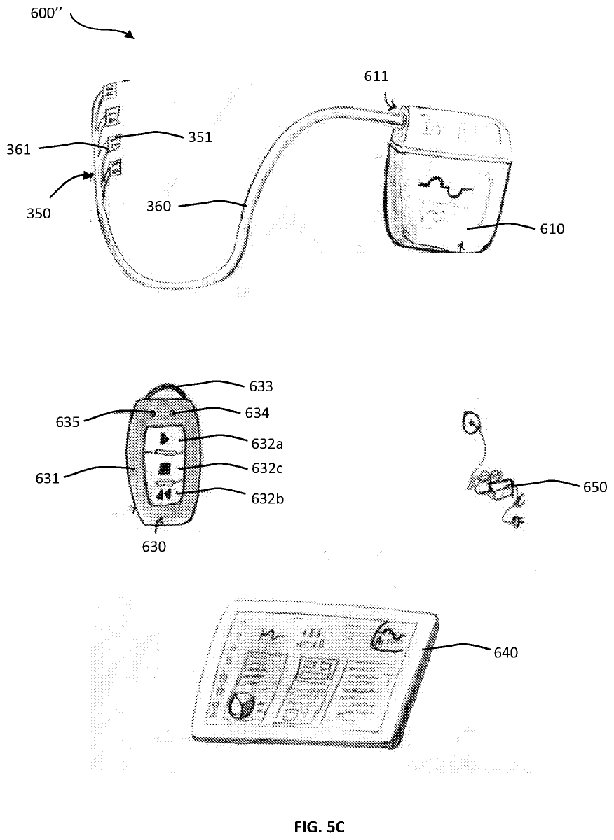

A system 600 according to an embodiment of the present disclosure, suitable for use in methods described herein, is shown in FIG. 5A. The system may comprise an electrode array 300 or 400 as described above. Optionally, the system 600 comprises a system controller 610. The system controller 510 may selectively trigger the applying of the activating stimulation signal 110 and blocking stimulation signal 120.

The system controller 610 may be configured to trigger the applying of the activating stimulation signal 110 and blocking stimulation signal 120 in response to an input from the patient, a caretaker or a clinician. For example, the system controller 610 may be selectively actuated by the patient, a caretaker or a clinician to initiate or cease the applying of the activating and blocking stimulation signals 110, 120 or to modify one or more parameters of the stimulating signal. The system controller 610 may also be configured to determine which of the first, second and third pairs of electrodes 310, 320, 330 (or 410, 420, 430) is selected to apply the activating stimulation signal 110, to apply the blocking stimulation signal 120 and to detect the evoked neural response 140. In this way, the system controller 610 may control whether the unidirectional stimulation produced is aVNS or eVNS.

The system controller 610 may receive signals from the electrode array 300, as indicated by the dotted line in FIG. 5. For example, the system controller 610 may be configured to receive evoked neural response signals 140 detected by one or more of the electrode pairs 310, 320, 330 (or 410, 420, 430). The system controller 610 may be configured to adjust one or more parameters of the activating stimulation signal and/or the blocking stimulation signal in response to the detected evoked neural response signal 140. For example, the system controller 610 may be configured to automatically tune the activating stimulating signal 110 and/or the blocking stimulating signal 120 to each other, to ensure that the activating stimulating signal 110 is applied within a therapeutic window in which the evoked neural response 140 is effectively blocked (as discussed in further detail below).

In some embodiments, the system 600 further comprises a glucose sensor 620 adapted to detect a glycaemia of the patient. The glucose sensor 620 may be associated with the system controller 610, as indicated by the dot-dash line in FIG. 5. The system controller 610 may be configured to trigger the applying of the activating stimulation signal 110 and blocking stimulation signal 120, or to automatically adjust one or more parameters of the activating and/or blocking stimulation signals 110, 120, in response to a detected glycaemia of the patient. For example, the system controller 610 may be configured to trigger the production of eVNS stimulation, or reduce or stop eVNS stimulation, in response to a detected glucose level above a maximum threshold, or to trigger the production of aVNS stimulation, or reduce or stop aVNS stimulation, in response to a detected glucose level below a minimum threshold. The maximum and minimum thresholds may be set by, for example, a patient's healthcare representative.

Another system 600′ according to an embodiment of the present disclosure, suitable for use in methods described herein, is shown in FIG. 5B. The system 600′ is substantially identical to the system 600 discussed above with reference to FIG. 5A, except that it includes a patient interface 630 to receive an input from the patent, caretaker of clinician, e.g. to initiate or cease the applying of the activating and blocking stimulation signals 110, 120 or to modify one or more parameters of the stimulating signal, and further includes a diagnostics unit 640 unit. The diagnostics unit 640 may be provided as a software application on a desktop computer, laptop, tablet, smartphone or otherwise. The diagnostics unit 640 may be configured to receive, store and/or display data from the system controller 610 relating to the applied stimulation parameters, glycaemia of the patient over time, therapeutic outcomes or otherwise.

FIG. 5C provides an illustration of a system 600″ that is very similar to the system 600′ discussed above with reference to FIG. 5B, but includes an electrode array 350 having four pairs of electrodes, configured on respective cuff portions 351 each located on a respective branch portion 361 of a lead 360. The lead 360 is releasably connectable at a port 611 to the system controller 610, the system controller 610 being configured as a unit that has an outer housing that is at least partially implantable (e.g. subcutaneously) in the patient.

The patient interface 630 of the system 600″ includes a housing 631 and plurality of buttons 632a, 632b, 632c, that are at a surface of the housing, the buttons 632a, 632b, 632c being configured to receive the input(s) from the patient, caretaker or clinician. The patient interface 630 is a hand-held battery-powered unit in this embodiment, which includes a key ring loop 633 so that the patient interface may be configured to mount on a key ring, although other means of clipping the interface to clothing or supporting the interface on a patient (e.g. a lanyard) may also be provided. The patient interface 630 can communicate wirelessly with the system controller 610.

In this embodiment, the buttons 632a, 632b, 632c include a first button 632a to receive an input from a patient, caretaker or clinician to selectively trigger eVNS stimulation, a second button 632b to receive an input from a patient, caretaker or clinician to selectively trigger aVNS stimulation, and a third button 632c to receiving an input from a patient, caretaker or clinician to selectively cease any applying of the activating and blocking stimulation signals. The patient interface 630 also optionally includes one or more lights 634, 635 (e.g. LEDs) to indicate that the patient interface is active, the stimulation is active, the system is in an automatic or manual mode of operation, or otherwise. In this embodiment, the patient interface 630 is a relatively sturdy unit with buttons 632a, 632b, 632c that are sturdy so as to prevent accidental pressing and allow simple operation. Nevertheless, it will be recognised that the patient interface may be provide in a different form while having substantially the same function, e.g. by being provided as a software application on a desktop computer, laptop, tablet, smartphone, or otherwise.

The system, 600″ also includes an inductive charger 650 for charging one or more components of the system such as the system controller 610. In this embodiment the diagnostics unit 640 is provided on a tablet computer.

### Example 1

Male Sprague-Dawley rats at 8-10 weeks old were used for experimental studies. The rats were kept on a 12 hour light (7 am-7 pm)/dark cycle (7 pm-7 am) and allowed ad libitum access to fresh food, standard chow and water. Prior to the experiment, the rats were fasted overnight (14-16 hours). On the day of the experiment, the rats were anaesthetised (2-3% isoflurane using an oxygen flow rate of 1-1.5 L/min) and breathing rate kept between 45 to 60 breaths per minute. The rats were kept hydrated and blood loss replaced using sterile Hartman's solution. The rats were kept anaesthetised for the duration of the experiment.

Prior to a step of implantation, the rats were anaesthetised. Moreover, the ventral abdominal midline incised and the ventral oesophagus and sub-diaphragmatic anterior abdominal branch of the vagus nerve exposed. The vagus nerve was gently dissected away from the oesophagus and an electrode array of the type shown in FIG. 3 was implanted rostral to the hepatic and celiac branches of the vagus nerve.

The electrode array included first, second and third pairs of platinum (99.95%) electrodes (E1-E2, corresponding to electrode pair 310; E3-E4, corresponding to electrode pair 320; and E5-E6, corresponding to electrode pair 330) embedded into a medical grade silicone elastomer cuff. Each platinum electrode had an exposed surface area of 0.39 mm². The electrodes of each of the electrode pairs (e.g. E1 and E2) were positioned on opposite silicone cuff portions, off-set by 0.32 mm. A channel (0.55 mm wide×0.2 mm deep) traversed the length of the array, and was positioned around the vagus nerve such that, when implanted, the electrodes of each of the electrode pairs were positioned on opposite sides of the vagus nerve. The silicone cuff portion sutured closed to prevent the nerve from migrating out of the channel.

The distance between the centre of adjacent electrode pairs (i.e. E1-E2 to E3-E4, or E3-E4 to E5-E6) was 3.4 mm, and the distance between the centre of adjacent electrodes E1-E2 to E5-E6 was 6.8 mm. Individually insulated 25 μm diameter platinum/iridium (ratio of 90/10) wires welded to each electrode formed a helical cable which extended to a percutaneous connector mounted on the lumbar region of the rat.

A silicone anchor tab embedded with Dacron (polyethylene terephthalate) fibres, located adjacent to the electrode array, was sutured to the oesophagus to provide mechanical stabilisation. The abdominal cavity and skin were then sutured closed. The left and right femoral veins were exposed and cannulated.

At T=−5 minutes, baseline, control blood samples were taken. At T=0 minutes a bolus of glucose (500 mg/kg; i.v.) was administered and a series of blood samples were taken (at 5, 12, 30, 60 and 90 minutes post bolus delivery) for analysis of glucose and pancreatic hormones. 30 minutes following collection of the last blood sample, the experiment was repeated again, as a reverse cross over sequence control. For each rat, vagus nerve neuromodulation (as described in further detail below) was continually applied for a test period of 60 minutes. A further test with no stimulation acted as a control.

The functionality of the electrodes was tested by measuring the common ground impedance of the electrodes. Biphasic current pulses (25 μs per phase and current of 931 μA) were passed between the electrode of interest and all other implanted electrodes, and the peak voltage at the end of the first phase (Wₜₒₜₐₗ) measured. The Wₜₒₜₐₗ value was then used to calculate total impedance (Zₜₒₜₐₗ) using Ohm's law (Z=voltage/current). Electrically-evoked compound action potentials (ECAPs) were recorded to ensure appropriate stimulation was delivered. ECAPs were generated using electrode pair E5-E6 to stimulate (bipolar stimulation, 200 μs pulse width with 50 μs interphase gap; 10 Hz) and E1-E2 to record (bipolar recording). Two sets of evoked electrophysiological recordings (averaged from a total of 50 responses) were made at currents from 0 to 2 mA in 0.1 mA steps. Recordings were sampled at a rate of 100 kHz and filtered (high pass: 200 Hz; low pass: 2000 Hz; voltage gain 10²). An electrically-evoked neural response threshold was defined as the minimum stimulus intensity producing a response amplitude of at least 0.1 μV within a post-stimulus latency window of 5 ms to 15 ms. All experiments ensured that stimulation current was suprathreshold. All recorded neural responses had conduction velocities within the range of a C-fibre response.

An activating stimulation signal was applied to evoke a neural response (ECAP) in the vagus nerve. The activating stimulation signal was delivered within the pre-determined therapeutic window and at 200 μs per phase, 50 μs interphase gap at 15 pulses per second.

A blocking stimulation signal was applied (simultaneously with the activating stimulation signal) to produce a reversible, focal block on the vagus nerve with the purpose of achieving unidirectional stimulation of the vagus nerve.

The blocking stimulation signal comprised alternating current stimulation, having (i) a sinusoidal wave of 8 mA current (peak to peak) and 40 kHz frequency (sinusoidal wave blocking) or (ii) a square wave of 4 mA current (peak to peak) and 4 kHz frequency (square wave blocking), to produce a reversible focal block on the nerve.

Directional stimulation of afferent vagal fibres (aVNS), which selectively activated only afferent fibres signalling to the brain, was achieved by simultaneously applying the blocking stimulation signal at the second electrode pair (E3-E4), and applying the activating stimulation signal to the third electrode pair (E5-E6).

Directional stimulation of efferent vagal fibres (eVNS), which selectively activated only efferent fibres signalling to the pancreas, was achieved by simultaneously applying the blocking stimulation signal to electrode pair (E3-E4) and applying the activating stimulation signal to the first electrode pair (E1-E2).

The effectiveness of directional stimulation was validated by detecting the evoked neural response during focal nerve blocking (eVNS) to assess the neural threshold. As shown in FIG. 4, application of the blocking stimulation signal (sinusoidal wave) increases the threshold amplitude at which the activating stimulation signal pulses generated evoked compound action potentials (ECAPs) in the vagus nerve. In the left-hand pane (with no blocking stimulation applied), the threshold amplitude for generating an ECAP was 150 μA. Activating stimulation pulses applied below this minimum amplitude will not reliably generate an ECAP in the nerve. In the right-hand pane (with blocking stimulation signal applied), the threshold amplitude for generating an ECAP increased to 300 μA. The blocking stimulation signal can be seen to effectively inhibit transmission of neural responses evoked by activating stimulation signals below this maximum amplitude. In this example, therefore, the current level able to be delivered while being effectively blocked, to produce unidirectional stimulation, was between 150 μA-250 μA. The activating stimulation signal was applied within the lower half of this ‘therapeutic window’.

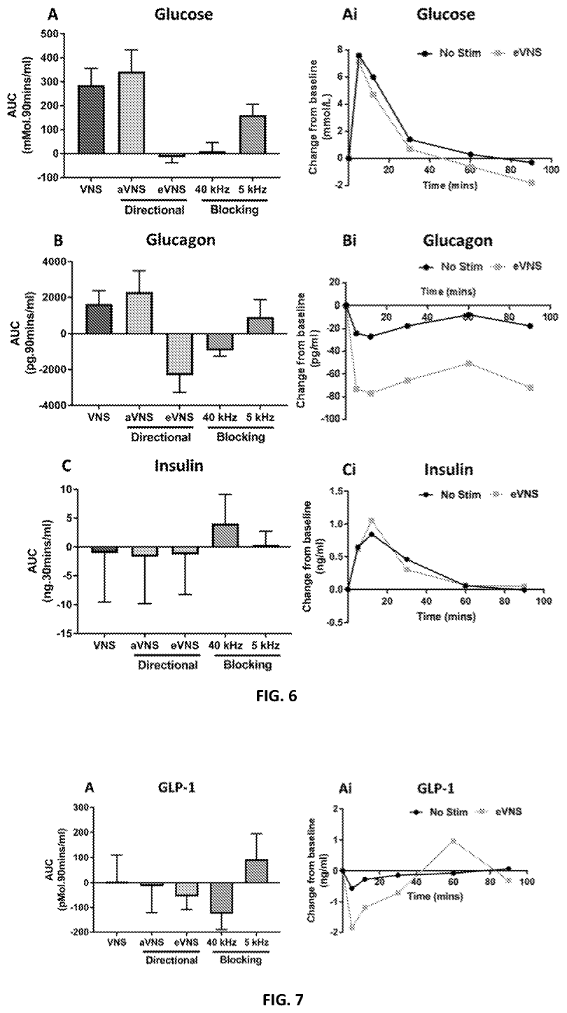

The results from the experimental procedure detailed above can be seen in FIGS. 6 to 11 as discussed below.

FIG. 6, shows the effect of eVNS on glucose and pancreatic hormone secretions in normal rats. A number of neuromodulation paradigms were applied continually for 60 minutes following a glucose bolus. These included vagal nerve stimulation (VNS; 15 Hz activating stimuli; n=6), aVNS (directionally activating afferent fibres only; n=11), eVNS (directionally activating efferent fibres only; n=11), 40 kHz (sinusoidal blocking stimuli; n=4) and 5 kHz (square pulse blocking stimuli; n=6). The graphs in panels A, B and C show the area under the curve (AUC) quantified for the glucose (A), glucagon (B) and insulin (C) responses. The data show mean AUC±standard error of mean. Panels Ai, Bi and Ci show representative traces of the typical eVNS response are for glucose (Ai), glucagon (Bi) and insulin (Ci).

FIG. 7 shows the effect of eVNS on the incretin hormone GLP-1 in normal rats. A number of neuromodulation paradigms were applied continually for 60 minutes following a glucose bolus. These include vagal nerve stimulation (VNS; 15 Hz activating stimuli; n=6), aVNS (directionally activating afferent fibres only; n=6), eVNS (directionally activating efferent fibres only; n=6), 40 kHz (sinusoidal blocking stimuli; n=3) and 5 kHz (square pulse blocking stimuli; n=5). Panel A shows the area under the curve (AUC) quantified for the GLP-1 response. The data show mean AUC±standard error of mean. Panel Ai shows a representative trace of the typical eVNS response are for GLP-1.

The experimental protocol outlined above was repeated in two experimental models of diabetic rats. In the first model, rats were fed a high-fat diet for four weeks in combination with 35 mg/kg streptozotocin (STZ, intraperitoneal) as an accepted chemical model of late type II diabetes. In the second model, rats were fed a high-fat diet for 20 weeks as the most clinically relevant model of early type II diabetes. This model closely mimics diabetes induced by obesity and sedate lifestyle.

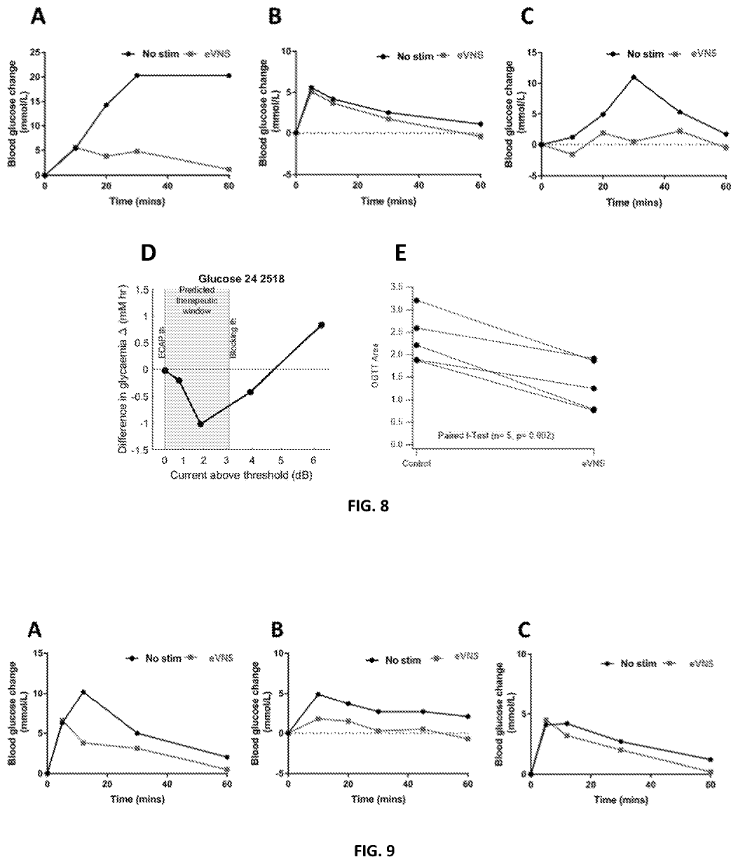

FIG. 8 shows the effect of eVNS in diet-induced diabetic rats. Panels A-C show the mean blood glucose (mmol/L) values for each rat during 60 minutes of continual eVNS (blocking stimulation signal: 8 mA, 25 kHz square pulse; activating stimulation signal 15 Hz and current applied within the pre-determined therapeutic window) following a bolus of glucose. As shown, when eVNS is applied, blood glucose is reduced compared to levels occurring when no stimulation is applied. Panel D shows a dose-response curve wherein different levels of eVNS stimulation within the pre-determined therapeutic window (0, 1 and 2 dB above the threshold amplitude for generating an ECAP) and above the pre-determined therapeutic window (4 and 6 dB above the threshold amplitude for generating an ECAP) were applied. As shown, the maximum reduction in blood glucose occurred with stimulation 2 dB above the threshold for this example. Panel E shows blood glucose reductions resulting from eVNS stimulation 2 dB above the threshold amplitude for generating an ECAP for 5 of the rats.

FIG. 9 shows the effect of eVNS in chemically-induced diabetic rats. Panels A-C show the mean blood glucose (mmol/L) values for each rat during 60 minutes of continual eVNS (blocking stimulation signal: 8 mA, 25 kHz square pulse; activating stimulation signal 15 Hz) following a bolus of glucose. As shown, when eVNS is applied, blood glucose is reduced compared to levels occurring when no stimulation is applied.

### Example 2

Two electrode arrays, each comprising two pairs of electrodes were implanted in a rat at the abdominal vagus nerve and the cervical vagus nerve, respectively. At the abdominal vagus nerve electrode array, a 15 Hz activating stimulation signal was applied at one electrode pair in combination with either a 40 kHz, 8 mA peak to peak sinusoidal blocking stimulation signal or a 4 kHz, 4 mA peak to peak, 90 μs square pulse blocking stimulation signal applied at the other electrode pair. Bipolar ECAP detection and recording was performed at the cervical vagus nerve electrode array to confirm activation of neural fibres and/or the degree of blocking.

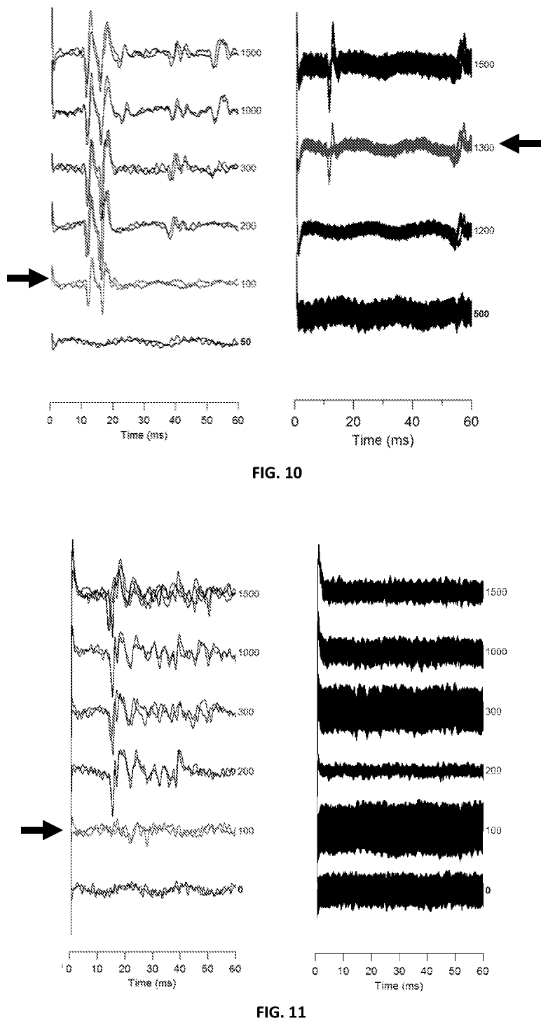

The results from the procedure detailed above are shown in FIGS. 10 and 11. Application of a sinusoidal wave blocking stimulation signal increased the neural threshold, resulting in a partial block. By contrast, application of a square wave blocking stimulation signal prevented transmission of ECAPs, resulting in a full block.

FIG. 10, left hand panel, shows representative traces produced when activating stimulation was applied in the absence of a blocking stimulation signal. As indicated by the left-hand arrow, the threshold for generating an neural response in the absence of a blocking stimulation signal was 100 μA. FIG. 10, right-hand panel, shows representative traces from the same rat when sinusoidal blocking stimulation was applied simultaneously with the activating stimulation. As indicated by the right-hand arrow, the threshold increased to 1300 μA (increased by a factor of 13). That is, the blocking stimulation signal blocked the transmission of evoked neural responses past the blocking location up to an amplitude of 1300 μA.

It was found that a sinusoidal blocking stimulation signal with a frequency of 1000 Hz or 5000 Hz had little discernible effect on the threshold amplitude, whereas a blocking stimulation signal with a frequency of 10000 Hz increased the evoked potential threshold by 2.5 dB and a blocking stimulation signal with a frequency of 26000 Hz increased the evoked potential threshold by 1.1 dB.

By contrast, as shown in FIG. 11, application of the square pulse blocking stimulation signal resulted in elimination of all ECAPs, resulting in a full block of the nerve. That is, no evoked neural responses were detected.

It was concluded that a blocking stimulation signal with a square pulse waveform may be more effective at producing a full block of evoked neural responses than a blocking stimulation signal with a sinusoidal waveform.

In both cases (sinusoidal blocking and square blocking), the blocking effect was found to be localised. That is, the nerve could be directionally stimulated following application of blocking stimulation by applying a 15 Hz activating stimulation signal to an adjacent location of the nerve (activating stimulation was applied at an activating location, 3.85 mm away from the blocking location at which the blocking stimulation was applied). In each case, the neural threshold for generating ECAPs at the activating location were similar to activating thresholds derived in the absence of blocking, indicating that the blocking effect on the nerve was localised. Thus, the nerve could be directionally stimulated such that nerve firing propagated in one direction only, in this example, caudally, to produce eVNS.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for modulating glycaemia in a patient, the method comprising:
applying an activating stimulation signal at an activating location at the vagus nerve, the activating stimulation signal configured to evoke a neural response in the vagus nerve; and
applying a blocking stimulation signal at a blocking location at the vagus nerve, the blocking stimulation signal configured to inhibit transmission of the evoked neural response along the vagus nerve past the blocking location,
to produce unidirectional vagal nerve stimulation, the unidirectional vagal nerve stimulation being effective to modulate glycaemia in the patient.

2. The method of claim 1 wherein the unidirectional stimulation is afferent vagal nerve stimulation.

3. The method of any one of claim 1 wherein the unidirectional stimulation is efferent vagal nerve stimulation.

4. The method of any one of the preceding claims wherein the applying of the activating stimulation signal and blocking stimulation signal is at a sub-diaphragmatic portion of the vagus nerve.

5. The method of claim 4 wherein the applying of the activating stimulation signal and blocking stimulation signal is at the abdominal vagus nerve.

6. The method of any one of the preceding claims wherein the activating stimulation signal has a frequency of between about 1 Hz to about 50 Hz.

7. The method of any one of the preceding claims wherein the activating stimulation signal has a frequency of about 15 Hz.

8. The method of any one of the preceding claims wherein the blocking stimulation signal is configured to inhibit transmission of the evoked neural response at the blocking location whilst allowing one or more adjacent regions of the nerve to conduct.

9. The method of any one of the preceding claims wherein the blocking stimulation signal has a frequency of between about 10 kHz to about 150 kHz.

10. The method of claim 9 wherein the blocking stimulation signal has a frequency of between about 10 kHz to about 50 kHz.

11. The method of claim 10 wherein the blocking stimulation signal has a frequency of between about 10 kHz to about 40 kHz.

12. The method of claim 11 wherein the blocking stimulation signal has a frequency of about 25 kHz.

13. The method of claim 12 wherein the blocking stimulation signal has a frequency of about 40 kHz.

14. The method of any one of the preceding claims wherein the blocking stimulation signal has an amplitude of between about 1 mA to about 50 mA.

15. The method of any one of the preceding claims wherein a plurality of blocking stimulation signals are applied at a corresponding plurality of blocking locations at the vagus nerve, each blocking stimulation signal configured to inhibit transmission of the evoked neural response along the nerve past the respective blocking location, to produce the unidirectional vagal nerve stimulation.

16. The method of any one of the preceding claims, further comprising detecting the evoked neural response in the vagus nerve.

17. The method of claim 16, wherein one or more parameters of at least one of the activation and blocking signals is at least partially based on properties of the detected neural response.

18. The method of any one of the preceding claims comprising determining at least one of:
a minimum threshold amplitude of the activating stimulation signal to evoke a compound action potential response in the vagus nerve; and
a maximum threshold amplitude of the activating stimulation signal above which transmission of an evoked neural response along the vagus nerve is not effectively inhibited by the blocking stimulation signal.

19. The method of claim 18, comprising applying the activating stimulation signal at an amplitude that is within a therapeutic window defined between the minimum threshold amplitude and the maximum threshold amplitude.

20. The method of claim 19, comprising applying the activating stimulation signal at an amplitude that is in the lower half of the therapeutic window.

21. The method of claim 18, 19 or 20, comprising applying the activating stimulation signal at a signal amplitude that is within a predetermined range above, or at a predetermined level above, the minimum threshold amplitude.

22. The method of claim 21, wherein the predetermined range above the minimum threshold amplitude is 0 to 4 dB, 0 to 3 dB, 0.5 to 4 dB, 0.5 to 3.5 dB, 0.5 dB to 3 dB, 1 to 4 dB, or 1 to 3 dB, above the minimum threshold amplitude.

23. The method of claim 21, wherein the predetermined level above the minimum threshold amplitude is about 1.5 dB, about 2 dB, or about 2.5 dB above the minimum threshold amplitude.

24. The method of any one of the preceding claims, further comprising:
detecting glycaemia of the patient; and
selectively triggering the applying of the activating stimulation signal and the applying of the blocking stimulation signal in response to the detected glycaemia.

25. The method of claim 24,
wherein the unidirectional vagal nerve stimulation is configured to be afferent vagal nerve stimulation when the detected glycaemia is lower than a predetermined lower limit; and/or
wherein the unidirectional vagal nerve stimulation is configured to be efferent vagal nerve stimulation when the detected glycaemia is higher than a predetermined upper limit.

26. The method of any one of claims 24 to 25, wherein one or more parameters of at least one of the activation and blocking signals is at least partially based on properties of the detected glycaemia of the patient.

27. The method of any one of the preceding claims further comprising monitoring food intake of the patient and selectively triggering the applying of the activating stimulation signal and the applying of the blocking stimulation signal in response to food intake of the patient.

28. The method of any one of the preceding claims when used for treating or preventing a condition associated with impaired glucose regulation in the patient.

29. A system configured to perform the method of any one of claims 1 to 22, the system comprising:
a first pair of electrodes selectively operable for applying the activating stimulation signal; and
a second pair of electrodes selectively operable for applying the blocking stimulation signal.

30. A system configured to modulate glycaemia in a patient, the system comprising:
a first pair of electrodes selectively operable for applying an activating stimulation signal to the vagus nerve, the activating stimulation signal configured to produce an evoked neural response in the vagus nerve; and
a second pair of electrodes selectively operable for applying a blocking stimulation signal to the vagus nerve, the blocking stimulation signal configured to inhibit transmission of the evoked neural response along the vagus nerve past the second pair of electrodes.

31. The system of claim 30 wherein the first and second pairs of electrodes are spaced from each other by a distance A.

32. The system of claim 30 or 31, further comprising a third pair of electrodes selectively operable as a pair of detecting electrodes for detecting the evoked neural response.

33. The system of claim 32 wherein the second pair of electrodes is located intermediate the first and third pairs of electrodes and wherein the third pair of electrodes is spaced from the second pair of electrodes by a distance B.

34. The system of claim 33 wherein each of the first, second and third pairs of electrodes is selectively operable for applying the activating stimulation signal, for applying the blocking stimulation signal or for detecting the evoked neural response.

35. The system of any one of claims 30 to 34 further comprising a system controller for selectively triggering the applying of the activating and blocking stimulation signals.

36. The system of claim 35 wherein the system controller is configured to selectively trigger the applying of the activating and blocking stimulation signals in response to one or more of:
an input from the patient, a caretaker or a clinician; and
glycaemia of the patient detected by a glucose sensor.

37. The system of claim 36, wherein selectively triggering the applying of the activating and blocking stimulation signals comprises selectively triggering the applying of afferent vagal nerve stimulation or efferent vagal nerve stimulation.

38. The system of claim 36 or 37, wherein the system comprises a patient interface configured to receive the input from the patient, caretaker or clinician to selectively trigger the applying of the activating and blocking stimulation signals.

39. The system of claim 38, wherein the patient interface is configured to receive an input from the patient, caretaker or clinician to cease applying of the activating and blocking stimulation signals.

40. The system of claim 38 or 39, wherein the patient interface comprises one or more buttons to receive the input(s) from the patient, caretaker or clinician.

41. The system of any one of claims 38 to 40, wherein the patient interface is a hand-held battery-powered unit.

42. The system of claim 41, wherein the patient interface is configured to mount on a key ring.

43. The system of any one of claims 35 to 42 wherein the system controller is configured to adjust one or more parameters of the activating and blocking stimulation signals in response to one or more parameters of the evoked neural response and/or detected glycaemia of the patient.

44. The system of any one claims 30 to 43 wherein each pair of electrodes is comprised in an electrode array.

45. The system of claim 44 wherein the electrode array is adapted for placement on the vagus nerve such that the electrodes of at least one of the electrode pairs are positioned on opposite sides of the nerve.

46. The system of any one of claims 30 to 45 wherein at least one of the electrode pairs is comprised in an electrode mounting device, the electrode mounting device adapted to mount to the vagus nerve to electrically interface the at least one electrode pair with the vagus nerve.

47. The system of claim 46 wherein the electrode mounting device is adapted to clamp to the vagus nerve.

48. The system of claim 46 or 47 wherein the mounting device comprises at least one cuff, the electrodes of each of the electrode pairs being positioned on opposite sides of the cuff.

49. Use of the system of any one of claims 30 to 48 to produce unidirectional vagal nerve stimulation, the unidirectional vagal nerve stimulation being effective to modulate glycaemia in the patient.
